(19) United States
(12) Patent Application Publication
(10) Pub. No.: US 2023/0026095 A1
(43) Pub. Date: Jan. 26, 2023

(54) PROCESS FOR THE CONTINUOUS MANUFACTURE OF STATINS

(71) Applicant: DR. REDDY'S LABORATORIES LIMITED, Hyderabad (IN)

(72) Inventors: Jaganadha Rao VELAGA, Visakhapatnam (IN); Sahil GUPTA, Hyderabad (IN); Manohar VENKATACHALAM, Hyderabad (IN); Suhas JAWLEKAR, Hyderabad (IN); Srividya RAMAKRISHNAN, Hyderabad (IN); Naga Lakshmi Ramana SUSARLA, Hyderabad (IN); Rajeev Rehani BUDHDEV, Hyderabad (IN); Rakeshwar BANDICHHOR, Sultanpur (IN); Mohammed Yakoob SARDAR, Vizianagaram (IN); Jerome GNANAPRAKASAM, Hyderabad (IN); Ravi Kumar G, Srikakulam (IN)

(21) Appl. No.: 17/779,795

(22) PCT Filed: Nov. 27, 2020
(86) PCT No.: PCT/IB2020/061204

§ 371 Date: May 25, 2022

(30) Foreign Application Priority Data
Nov. 29, 2019 (IN) 201941049256

(51) Int. Cl. C07D 207/34

(52) U.S. Cl. CPC C07D 207/34; C07B 2200/13

(57) ABSTRACT

The present invention relates to process for the continuous manufacture of Statins or salts thereof. The present invention relates to process for the continuous manufacture of Atorvastatin or a salt thereof. The present invention relates to a continuous manufacturing process for the crystallization of Atorvastatin calcium. The present invention also relates to a continuous manufacturing process for the crystallization of Atorvastatin calcium Form I.

### FIELD OF THE INVENTION

The present invention relates to process for the continuous manufacture of Statins or salts thereof. The present invention relates to process for the continuous manufacture of Atorvastatin or a salt thereof. The present invention also relates to a continuous manufacturing process for the crystallization of Atorvastatin calcium.

### BACKGROUND OF THE INVENTION

Hydroxymethyl glutaryl Coenzyme A reductase (HMG-CoA) inhibitors, commonly called as Statins, are some of the most commonly prescribed medications worldwide. Statins are the selective inhibitors of HMG-CoA reductase enzyme, which converts 3-hydroxy-3-methylglutaryl-coenzyme to mevalonate, a precursor of sterols such as cholesterol. The conversion of HMG-CoA to mevalonate is an early and rate-limiting step in cholesterol biosynthesis. Evidence suggests that statin therapy has significant mortality and morbidity benefit for both primary and secondary prevention from cardiovascular disease. Currently, there are at least five statin drugs available on the market namely Atorvastatin (Lipitor®), Simvastatin (Zocor®), Rosuvastatin (Crestor®), Pitavastatin (Livalo®) and Fluvastatin (Lescol®). Globally Statins are marketed primarily as a lipid-lowering agent and for prevention of events associated with cardiovascular disease.

Various processes for the manufacture of statins and their intermediates are described in literature extensively in batch mode. Most of those methods for the manufacture of Statins proceed through the ester intermediate of formula II, which is described as a key convergent step followed by converting it to Statin or a salt thereof, wherein R1 and R2 are hydroxy protecting groups; R3 is alkyl or aryl and A corresponds to the remaining statin moiety.

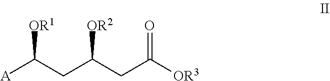

For such high-volume and commonly used statins drugs, there remains necessity to manufacture in a much productive yet benign methods to meet the needs of wide patient pool. Although there are various batch manufacturing methods of Statins reported in the literature, several challenges still exist, for example, the deprotection of intermediate of formula II of Atorvastatin, is typically achieved by use of acids like HCl, and process intensification demands for higher temperature leading to increased impurity formation which are extremely difficult to control in a batch mode. This introduces the need for multiple purifications which leads to increase in downstream operations such as filtrations, cake washings, and optionally drying, making the process tedious. The multiple purification steps also lead to consumption of additional solvents which makes the process environmentally non-friendly. Further, there are additional challenges associated with intermediates of Statins, such as filtration, drying and other downstream operations adding-up laborious steps. Each of these additional operation requires dedicated equipment leading to huge footprint and prolonged cycle times. In the reported batch processes, including one-pot approach wherein the isolation or purifications of intermediates are avoided, the typical limitations associated with a batch processes still remain. Most of the reported processes are still susceptible to formation of impurities and decrease in yield, due to the known sensitivity of Statin to temperature and harsh acid or base environment. Ironically, without the use of harsh acid catalysts or bases the process intensification remains incomplete.

The inventors of the instant application have developed a robust process for manufacture of Statins from the intermediate of formula II, which adopts an intensified in-situ continuous manufacturing process, eliminating the several limitations associated with batch processes. The continuous process results in shorter reaction times with minimal solvent consumption, making the process environmentally benign, removes the need for multiple unit operations making the set up modular and drastically reducing the footprint. The inventors have demonstrated the continuous process is capable of delivering high quality product, meeting all the required specifications consistently and is commercially viable for practice at an industrial scale.

### SUMMARY OF THE INVENTION

In an aspect, the present application provides a continuous manufacturing process for the preparation of a Statin or salt thereof, comprising the steps of:
I. contacting a stream containing the compound of formula II in an inert solvent with an acid stream in a flow reactor, to form compound of formula III;

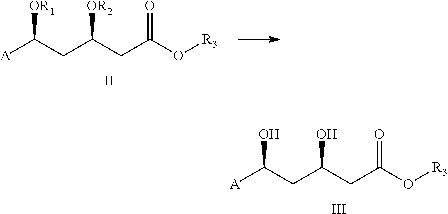

II. converting the compound of formula III to Statin or a salt thereof; wherein R¹ and R² may be same or different and are H, C₁-C₆ alkyl, C₁-C₆ alkoxy, silyl or R¹ and R² together are CR^{a}R^{b} wherein R^{a} and R^{b} may be same or different and are independently an C₁-C₁₁ alkyl group or R^{a} and R^{b}, together with the carbon atom to which they are attached, may form a ring, and R³ is an C₁-C₆ alkyl group; and A is selected from the groups consisting of:

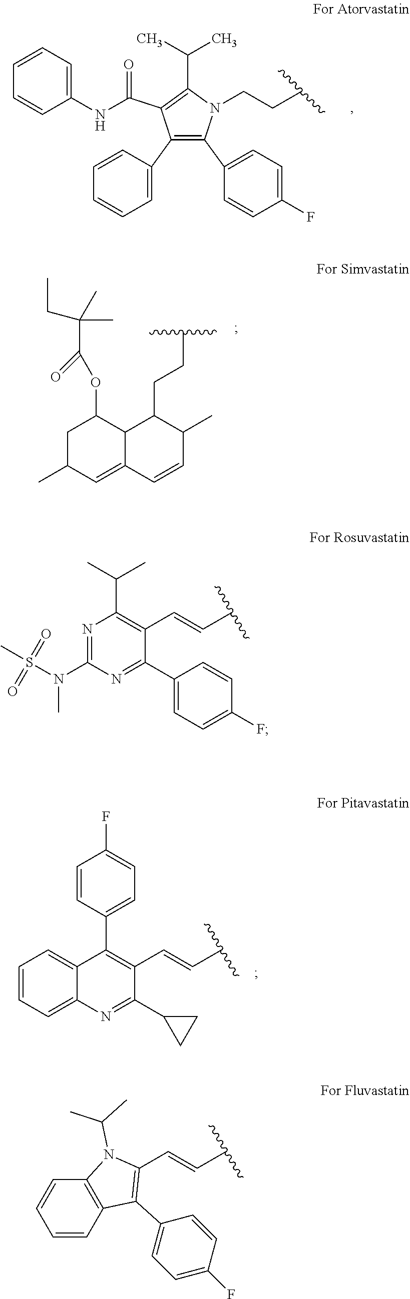

In another aspect, the present application provides a continuous manufacturing process for the preparation of Atorvastatin or a salt thereof, comprising the steps of:
I. contacting a stream containing the compound of formula IIa in an inert solvent with an acid stream in a flow reactor, wherein R¹ and R² may be same or different and are C₁-C₆ alkyl, C₁-C₆ alkoxy, silyl or R¹ and R² together are CR^{a}R^{b} wherein R^{a} and R^{b} may be same or different and are independently an C₁-C₁₁ alkyl group or R^{a} and R^{b}, together with the carbon atom to which they are attached, may form a ring, and R³ is an C₁-C₆alkyl group, to form compound of formula IIIa;

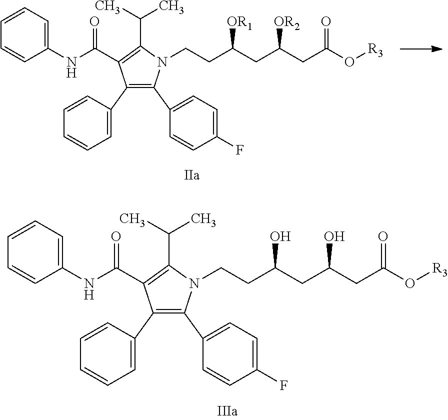

III. converting the compound of formula IIIa to Atorvastatin or a salt thereof.

In another aspect, the present application provides a continuous manufacturing process for the preparation of Atorvastatin calcium comprising the steps of:
I. contacting a stream containing compound of formula IIa in an inert solvent with an acid stream in a flow reactor, wherein R¹ and R² may be same or different and are C₁-C₆ alkyl, C₁-C₆ alkoxy, silyl or R¹ and R² together are CR^{a}R^{b} wherein R^{a} and R^{b} may be same or different and are independently an C₁-C₁₁ alkyl group or R^{a} and R^{b}, together with the carbon atom to which they are attached, may form a ring, and R³ is an C₁-C₆ alkyl group, to form compound of formula IIIa;

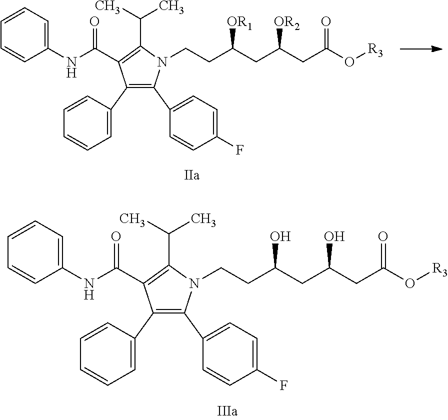

II. contacting the stream containing compound of formula IIIa of step I with a stream containing source of cation M in a flow reactor, where in the cation is selected from the group consisting of Sodium, Potassium and Calcium, to form the corresponding salt of atorvastatin;

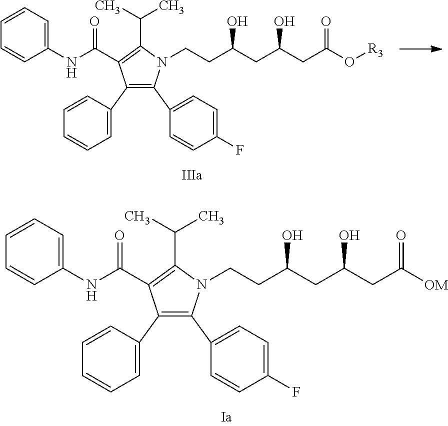

III. when M of step II is other than Calcium ion, contacting the stream containing salt of Atorvastatin of step II with a source of calcium ion in the flow reactor, where in the source of calcium ion is selected from the group consisting of calcium acetate, calcium carbonate and calcium chloride, calcium bromide to form atorvastatin calcium;

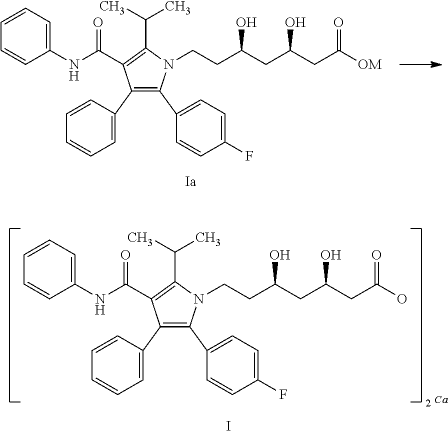

IV. crystallizing atorvastatin calcium formed at step II or III, optionally in the presence of seed crystals.

In another aspect, the present application provides a continuous manufacturing process for the crystallization of Atorvastatin calcium, comprising the steps of:
I. providing a solution stream containing Atorvastatin calcium in an organic solvent;
II. crystallizing Atorvastatin calcium in flow reactor.

In another aspect, the present application provides a continuous manufacturing process for the crystallization of Atorvastatin calcium Form I, comprising the steps of:
I. providing a solution stream containing Atorvastatin calcium in an organic solvent;
II. contacting the solution stream of step I with a stream of anti-solvent, optionally containing seeds of Form I, in flow reactor.

### BRIEF DESCRIPTION OF THE DRAWING

FIG. 1 is an XRPD of the crystalline Form I of Atorvastatin calcium prepared according to example 16.

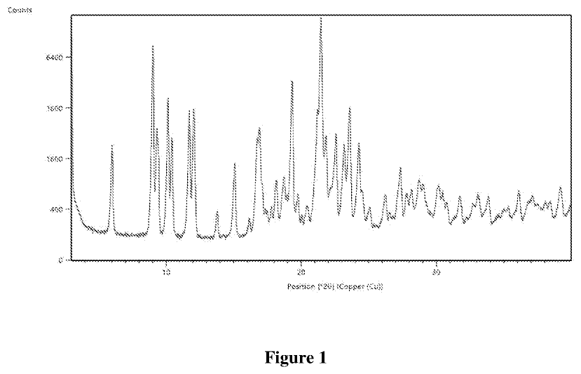

### DETAILED DESCRIPTION OF THE INVENTION

Aspects of the present application provide a processes for the continuous manufacture of Statins or a salt thereof.

The process for the continuous manufacturing Statins is carried out in flow reactors. A flow reactor is a system through which the reactants are continuously pumped and product continuously collected. It is typically a device containing microchannels, static mixers or dynamic mixers which aids in enhanced heat and mass transfer properties. Further the reactors can be divided into static and dynamic mixing reactors, where the static reactors do not have any moving parts whereas the dynamic reactors have moving parts. The narrow channels in the reactors result in enhanced mixing resulting in near-plug flow conditions, thereby removing the mass transfer limitation typically associated with batch reactors. The flow reactors are built to withstand high temperature and pressure conditions, thus allowing for exploration and execution of novel process windows which require operation at high temperatures. The reactors are also small in size, occupying less footprint and allowing for operating flexibility.

The advantages of the continuous process presented in the current application involves an intensified process where the reaction times are precisely controlled down to a few minutes or less. In particular the deprotection of intermediate compound of formula II with an acid, such as hydrochloric acid, is achieved by operating at temperatures well above the boiling point of the solvents enhancing reaction rates to be faster than the batch conditions. Similarly the reaction times of hydrolysis and salt exchange reactions are also reduced by identifying a design space which is a combination of residence time, reagent concentration and operating temperatures. Next, the crystallization operation is carried out in a continuous manner, via a combination of a dynamic mixing reactor and CSTRs, followed by continuous filtration and drying. Thus making it an end to end continuous manufacturing for Statins or a salt thereof. Further, the scalability of the developed process is also demonstrated. The process presented in the present invention lead to robust process with significant reduction in reactions times due to enhanced kinetics, thereby leading to reduction in number of unit operations, while achieving consistent quality attributes.

TABLE 1
Comparison of one pot batch process vs flow process

| | | | | | Time required for | | | |
|---|---|---|---|---|---|---|---|---|
| | | | | | generation of ~300 kg | | | |
| | | | | | output (including | | | |
| | | | | | downstream operations | | | |
| | Reaction time | | | | till drying) | | | |
| | Batch | | Flow | | Batch | | Flow | |
| Reaction type | process | | process | | process | | process | |
| Deprotection of | 9 | h | 45 | s | ~100 | h | 24 | h |
| Compound of | | | | | | | | |
| formula IIa | | | | | | | | |
| Hydrolysis of ester | 2 | h | 120 | s | | | | |
| of formula IIa, to | | | | | | | | |
| obtain Atorvastatin | | | | | | | | |
| sodium salt | | | | | | | | |
| Salt exchange to | 1 | h | 120 | s | | | | |
| form Atorvastatin | | | | | | | | |
| calcium | | | | | | | | |

For the deprotection reaction in batch process, conversion of compound of formula IIa with time is depicted in below graph:
The process of present invention can produce Atorvastatin calcium at a rate of about 12.5 kg per hour with planned commercial scale equipment. The cycle time reduction via proposed flow process is significant when compared with the optimized one-pot batch process. As can be seen from Table-1, batch process takes about ˜100 h to generate 300 Kg as against 24 h through a flow process (including all downstream operations)

In an aspect, the present application provides a continuous manufacturing process for the preparation of a Statin or salt thereof, comprising the steps of:
I. contacting a continuous stream containing the compound of formula II in an inert solvent with an acid stream in a flow reactor, to form compound of formula III;

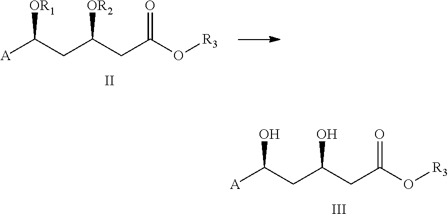

II. converting the compound of formula III to Statin or a salt thereof; wherein R¹ and R² may be same or different and are H, C₁-C₆ alkyl, C₁-C₆ alkoxy, silyl or R¹ and R² together are CR^{a}R^{b} wherein R^{a} and R^{b} may be same or different and are independently an C₁-C₁₁ alkyl group or R^{a} and R^{b}, together with the carbon atom to which they are attached, may form a ring, and R³ is an C₁-C₆ alkyl group; and A is selected from the group consisting of:

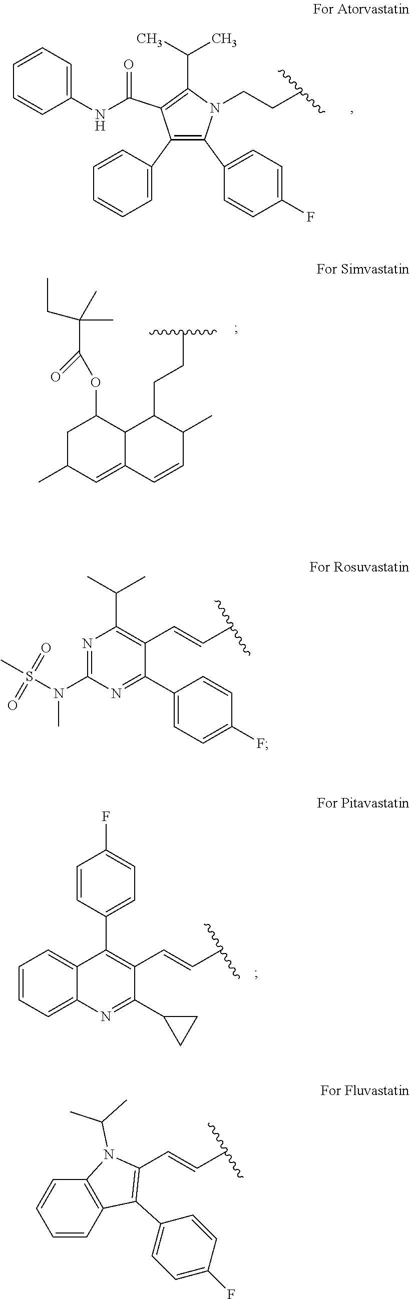

The starting material, compound of formula II may be procured from the existing commercial sources or synthesized according to any of the processes reported in the literature. Compound of formula II may be optionally purified according to methods known in the art such as recrystallization or any other separation techniques including chromatography, before using in any aspects of present application.

In embodiments, at least one step of the present aspect may be carried out in suitable flow reactors connected in the order of the reaction sequentially. Flow reactors may be selected from the group consisting of plug flow reactors (PFR), microreactors, dynamic mixing reactors such as dynamic agitated tubular reactor (ATR), dynamic agitated cell reactor (ACR), oscillatory baffled reactor/crystallizer (OBR/OBC), and the like.

In embodiments, the streams containing the reactants at each step are mixed under suitable conditions in suitable mixers which include but not limited to T-mixer, Y-Mixer, static mixer and micromixer and the like to achieve desired reaction. In embodiments, the streams containing the reactants may be supplied from pre-heated or pre-cooled reactors, based on the reaction conditions.

In embodiments, the flow reactors used in the process may be obtained from commercially available sources such as Corning®, Chemtrix®, AM Technologies® or customized reactors may be employed to achieve desired residence time and mixing of the reactants. In embodiments, an additional flow reactor may be included for prolonged residence time in the sequence. In embodiments, mixing can be enhanced through the insertion of static elements.

In embodiments, the flow rates of the streams containing individual or mixtures of raw materials may be controlled to achieve the desired residence time for the formation of product. The streams containing individual or mixtures of components may be in either heterogeneous or homogenous state.

In embodiments, step I of this aspect may be carried out by contacting the stream containing compound of formula II with the stream containing an acid to obtain a compound of formula III in a flow reactor. In embodiments, the flow reactors which can withstand a very high acidic/basic and high temperature and pressure environment are suitable for step I. In embodiments, flow reactors with high heat transfer coefficient and thermal conductivity are suitable for step I for effective heat transfer and to avoid thermal degradation of reactants. Suitable flow reactors for step I include, but not limited to Silicon carbide flow reactors, which has high thermal conductivity of 100 W/m K, from commercial brands such as Chemtrix® and Corning®. In preferred embodiments, the suitable silicon carbide flow reactors from Chemtrix, such as Protrix® for lab scale or Plantrix® for large scale.

In embodiments the streams may be contacted at a suitable temperature of about 70° C. or above. In preferred embodiment, the streams may be contacted between 70° C. and 100° C. In embodiments, the individual streams of step I are maintained at the desired temperature in a preheated reactors before mixing with each other. In embodiments, the residence time of the reaction mixture of step 1 is maintained for at least 30 seconds. In preferred embodiment, the residence time of the mixture is maintained for 30 to 180 seconds.

In embodiments, an acid of step I may be selected from the group consisting of organic and inorganic acids. Organic acids include, but not limited to formic acid, acetic acid, trifluoro acetic acid, methane sulfonic acid and the like. Inorganic acid include, but not limited to hydrochloric acid, sulfuric acid, nitric acid and the like. In embodiments, at least 1 equivalent of the acid is used in step I. In particular embodiments, about 1.5 to 3 equivalents are used, when HCl is the acid.

In embodiments, the individual streams of step I are present in homogeneous state by dissolving the compound of formula II or acid in a suitable inert solvent. Inert solvent may be selected from the group consisting of water, methanol, ethanol, 2-propanol, acetonitrile any other water-miscible organic solvent and mixture thereof.

In embodiments of this aspects, the step II of this aspect, for converting compound of formula II to Statin or a salt thereof, may be carried out according to methods described in any aspect of the present application or according to any alternate procedures known in the art. In embodiments, at least one step of the process for converting compound of formula II to Statin may be carried out in a flow reactor.

In embodiments, the statins or a salt thereof obtained according to the process of this aspect may be selected from the group consisting of Atorvastatin, Rosuvastatin, Simvastatin, Pitavastatin and Fluvastatin.

In another aspect, the present application provides a continuous manufacturing process for the preparation of Atorvastatin or a salt thereof, comprising the steps of:
I. contacting a stream containing the compound of formula IIa in an inert solvent, with an acid stream in a flow reactor, wherein R¹ and R² may be same or different and are C₁-C₆ alkyl, C₁-C₆ alkoxy, silyl or R¹ and R² together are CR^{a}R^{b} wherein R^{a} and R^{b} may be same or different and are independently an C₁-C₁₁ alkyl group or R^{a} and R^{b}, together with the carbon atom to which they are attached, may form a ring, and R³ is an C₁-C₆ alkyl group, to form compound of formula IIIa;

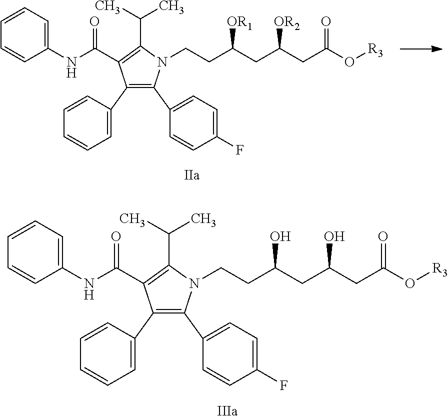

II. converting the compound of formula IIIa to Atorvastatin or a salt thereof.

In embodiments, step I of this aspect may be carried out by contacting the stream containing compound of formula IIa with the stream containing an acid to obtain a compound of formula IIIa in a flow reactor. In embodiments, the flow reactors which can withstand a very high acidic/basic and high temperature and pressure environment are suitable for step I. In embodiments, flow reactors with high heat transfer coefficient and thermal conductivity are suitable for step I to avoid thermal degradation of reactants. Suitable flow reactors for step I include, but not limited to Silicon carbide flow reactors, which has high thermal conductivity of 100 W/m K, from commercial brands such as Chemtrix® and Corning®. In preferred embodiments, the suitable silicon carbide flow reactors from Chemtrix, such as Protrix® for lab scale or Plantrix® for large scale.

In embodiments the streams may be contacted at a suitable temperature of about 70° C. or above. In preferred embodiment, the streams may be contacted between 70° C. and 100° C. In embodiments, the individual streams of step I are maintained at the desired temperature in a preheated reactors before mixing with each other. In embodiments, the residence time of the reaction mixture of step 1 is maintained for at least 30 seconds. In preferred embodiment, the residence time of the mixture is maintained for 30 to 180 seconds.

In embodiments, an acid of step I may be selected from the group consisting of organic and inorganic acids. Organic acids include, but not limited to formic acid, acetic acid, trifluoro acetic acid, methane sulfonic acid and the like. Inorganic acid include, but not limited to hydrochloric acid, sulfuric acid, nitric acid and the like. In embodiments, at least 1 equivalent of the acid is used in step I. In particular embodiments, about 1.5 to 3 equivalents are used, when HCl is the acid.

In embodiments, the individual streams of step I are present in homogeneous state by dissolving the compound of formula IIa or acid in a suitable inert solvent. Inert solvent may be selected from the group consisting of water, methanol, ethanol, 2-propanol, acetonitrile any other water-miscible organic solvent and mixture thereof.

In embodiments of this aspects, the step II of this aspect, for converting compound of formula IIIa to Atorvastatin may be carried out according to methods described in any aspect of the present application or according to any alternate procedures known in the art.

In another aspect, the present application provides a continuous manufacturing process for the preparation of Atorvastatin calcium comprising the steps of:
I. contacting the stream containing compound of formula IIa in an inert solvent, with an acid stream in a flow reactor, wherein R¹ and R² may be same or different and are C₁-C₆ alkyl, C₁-C₆ alkoxy, silyl or R¹ and R² together are CR^{a}R^{b} wherein R^{a} and R^{b} may be same or different and are independently an C₁-C₁₁ alkyl group or R^{a} and R^{b}, together with the carbon atom to which they are attached, may form a ring, and R³ is an C₁-C₆alkyl group, to form compound of formula IIIa;

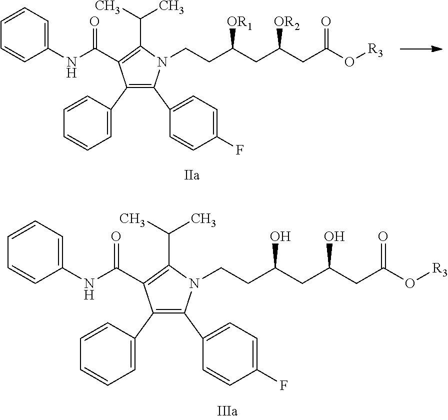

II. contacting the stream containing compound of formula IIIa of step I with the stream containing source of cation M in a flow reactor, where in the cation is selected from the group consisting of Sodium, Potassium and Calcium; to form the corresponding salt of atorvastatin.

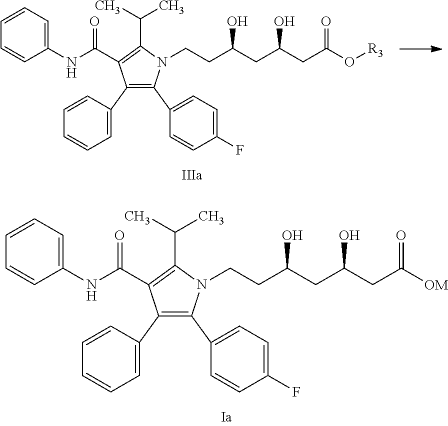

III. when M of step II is other than Calcium ion, contacting the stream containing salt of Atorvastatin of step II with a source of calcium ion in the flow reactor, where in the source of calcium ion is selected from the group consisting of calcium acetate, calcium carbonate and calcium chloride, calcium bromide; to form atorvastatin calcium;

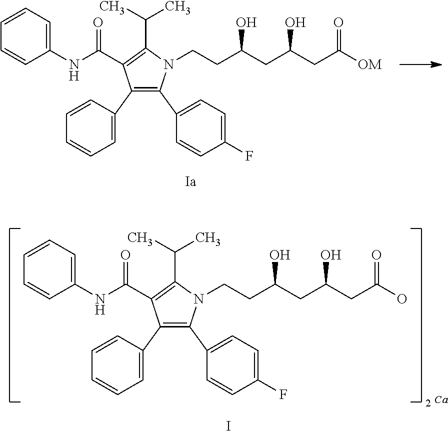

IV. crystallizing atorvastatin calcium formed at step II or III, optionally in the presence of seed crystals.

In embodiments, step I of this aspect may be carried out according to the procedures described in the previous aspect.

In embodiments, step II of the present aspect may be carried out by contacting the stream containing the compound of formula IIIa, obtained in step I, with the stream containing the source of cation in a flow reactor to obtain the corresponding salt of atorvastatin. Any suitable plug flow reactor may be used to carry out step II of this aspect.

In embodiments, step II of this aspect may be carried out by contacting the stream containing compound of formula IIIa with the stream containing the source of cation in a flow reactor at a suitable temperature of about 30° C. or above. In preferred embodiment, the streams may be contacted between 50° C. and 100° C. In embodiments, the stream containing the source of cation is maintained at the desired temperature in a preheated reactor before mixing. In embodiments, the residence time of the reaction mixture of step II is maintained for at least 50 seconds. In preferred embodiments, the mixture is maintained for 50 to 240 seconds.

Suitable flow reactors for step II include, but not limited to Silicon carbide flow reactors, from commercial brands such as Chemtrix® and Corning®. In preferred embodiments, the suitable silicon carbide flow reactors from Chemtrix, such as Protrix® for lab scale or Plantrix® for ton scale.

Cation may be selected from the group consisting of sodium, potassium and calcium. Cation sources may include, but not limited to corresponding hydroxides, carbonates or bicarbonates, chlorides, acetates. In embodiments, at least 1 equivalents of cation source is used at step II. In preferred embodiments, 3 to 5 equivalents of cation source is used, when the cation is other than calcium.

In embodiments, compound of formula IIIa may be mixed with a source of cation, which may in the homogeneous solution state by dissolving the cation source in a suitable inert solvent selected from the group consisting of water, any water miscible organic solvent like methanol, ethanol, acetone and mixture thereof.

In embodiments, step III of the present aspect may be carried out, when the cation of step II is other than Calcium ion, by contacting the stream containing salt of Atorvastatin obtained in step II with a stream containing source of calcium ion in a flow reactor to obtain atorvastatin calcium. Any suitable plug flow reactor or tubular reactor, preferably with dynamic tubular reactors may be used to carry out step II of this aspect. In preferred embodiments, dynamic mixing reactors such as Agitated tubular reactor (ATR) or Rotating tubular reactor (RTR) may be used to carry out step III of this aspect.

In embodiments, the source of calcium ion at step III may be selected from the group consisting of calcium acetate, calcium carbonate, calcium chloride and the like.

In embodiments, the stream containing salt of Atorvastatin obtained in step II may be contacted with a stream containing source of calcium ion to form atorvastatin calcium at a suitable temperature of about 50° C. or above. In preferred embodiments, the streams may be contacted between 50° C. and 100° C. In more preferred embodiments, the streams may be contacted between 55° C. and 75° C. In embodiments, the stream containing the source of cation is maintained at the desired temperature in a preheated reactor before mixing. In embodiments, the reaction mixture of step III is maintained for a residence time of at least 60 seconds or more. In preferred embodiments, the residence time is maintained at least for 90 seconds or more. In embodiments, about 0.55 equivalents of calcium source is used at step III. In preferred embodiments, 0.6 to 1 equivalents of calcium source is used.

In embodiments, the source of calcium ion may be in homogeneous solution state by dissolving in a suitable solvent selected from the group consisting of water, any water miscible organic solvent like methanol, ethanol, acetone and mixture thereof.

In embodiments, step IV of the present aspect may be carried out by crystallizing Atorvastatin calcium in flow reactor. Any suitable flow reactor may be used to carry out step IV of this aspect such as dynamic mixing reactors. In preferred embodiments, dynamic tubular or cell flow reactors such as Agitated cell reactor (ACR), Agitated tubular reactor (ATR), Rotating tubular reactor (RTR), Continuous stirred tank reactors (CSTR)/Mixed suspension mixed product removal (MSMPR) and Oscillatory baffled reactor/crystallizer (OBR/OBC) may be used to carry out step IV of this aspect.

Atorvastatin calcium may be crystallized out by any methods known in the art to relieve super saturation such as reducing the temperature of the stream containing Atorvastatin calcium, contacting with anti-solvent stream or continuous evaporation of the solvent. In embodiments, crystallization of atorvastatin calcium may be carried out in the presence of seed crystals.

In embodiments, the solution stream containing atorvastatin calcium may be contacted with anti-solvent. The suitable anti-solvent that may be used is a solvent in which Atorvastatin calcium is least soluble or insoluble, such as water. In embodiments, the continuous stream containing of the seed crystals in a suitable anti-solvent may be contacted with solution stream containing atorvastatin calcium. In embodiments, the individual streams may be contacted at a suitable temperature of about 0° C. or above. In embodiments, mixture may be maintained for longer residence time through the introduction of addition flow reactors to obtain the desired quality and quantity of solids.

In alternate embodiments, crystallization of atorvastatin calcium may be carried out by reducing the temperature of the mixture containing atorvastatin calcium. In embodiments, solution of atorvastatin calcium may be cooled prior to or after addition of seed crystals.

In embodiments, the solid atorvastatin calcium may be separated continuously using methods known in the art such as filtration or centrifuging. The separated solids may be dried continuously in a suitable drying conditions. Drying may be carried under air drying, vacuum drying, fluidized bed drying and the like. Drying may be carried out at a suitable temperature of about 25° C. or above for residence time of about 3 minutes or more.

Atorvastatin calcium obtained by any of the processes described in this application may be any crystalline form known in the art. In embodiments, Atorvastatin calcium obtained by any of the processes described in this aspect may be crystalline Form I.

In another aspect, the present application provides a continuous manufacturing process for the crystallization of Atorvastatin calcium, comprising the steps of:
I. providing a solution stream containing Atorvastatin calcium in an organic solvent;
II. crystallizing Atorvastatin calcium, in flow reactor.

In embodiments of these aspects, the solution stream containing the Atorvastatin calcium may be obtained by dissolving Atorvastatin calcium in organic solvents or by directly taking solution obtained from the synthetic process according any of the process of previous aspects or any other know procedures to make atorvastatin calcium. Atorvastatin calcium may be dissolved at suitable temperatures, optionally under heating. The solution may be made particle free through filtration and optionally treated with decolorizing agents or active carbon.

In embodiments, crystallizing Atorvastatin calcium from the solution of step I may be carried out by reducing the temperature of the solution containing Atorvastatin Calcium or by contacting with an anti-solvent or by removing the solvent through evaporation, under suitable conditions.

In embodiments, the organic solvent is selected from group consisting of methanol, ethanol, 2-propanol, any other water-miscible organic solvent and mixture thereof.

In embodiments, the anti-solvent is a solvent in which Atorvastatin calcium is insoluble or has very low solubility, such as water. In embodiments, the anti-solvent may be a suspension of Atorvastatin calcium seed crystals in water.

In embodiments, crystallization of Atorvastatin calcium may be carried out by reducing the temperature of the mixture containing atorvastatin calcium. In embodiments, solution of atorvastatin calcium may be cooled prior to or after addition of seed crystals. The solution of atorvastatin calcium may be cooled to a suitable temperature. In embodiments, mixture may be maintained for suitable residence time through the introduction of addition flow reactors to obtain the desired quality and quantity of solids.

In embodiments, step II of this aspect may be carried out using dynamic tubular or cell flow reactors with high wall shear selected from the group consisting of Agitated cell reactor (ACR), Agitated tubular reactor (ATR), Rotating tubular reactor (RTR), Oscillatory baffled reactor/crystallizer (OBR/OBC) and Continuous stirred tank reactors (CSTR).

In embodiments, the solid atorvastatin calcium may be separated continuously using methods known in the art such as filtration or centrifuging. The separated solids may be dried continuously in a suitable drying conditions. Drying may be carried under air drying, vacuum drying, fluidized bed drying and the like. Drying may be carried out at a suitable temperature of about 25° C. or above for residence time of about 3 minutes or more. In embodiments, drying may be carried out in a suitable drying equipment, preferably continuous drying equipment such as continuous air dryers, continuous vacuum dryers or continuous fluidized bed dryers.

Atorvastatin calcium obtained by any of the processes described in this application may be any crystalline form known in the art. In embodiments, Atorvastatin calcium obtained by any of the processes described in this aspect may be crystalline Form I.

In another aspect, the present application provides a continuous manufacturing process for the crystallization of Atorvastatin calcium Form I, comprising the steps of:
I. providing a solution stream containing Atorvastatin calcium in an organic solvent;
II. contacting the solution stream of step I with a stream of anti-solvent, optionally containing seeds of Form I, in flow reactor.

In embodiments, step I, of this aspect may be carried out according the procedures described for step I of the previous aspect.

In embodiments, the solution stream containing Atorvastatin calcium may be contacted with anti-solvent. The suitable anti-solvent that may be used is a solvent in which Atorvastatin calcium is least soluble or insoluble, such as water.

In embodiments, the anti-solvent may be a suspension of Form I seed crystals in water.

In embodiments, the stream of anti-solvent containing of the seed crystals may be contacted with solution stream containing atorvastatin calcium. In embodiment, ratio of anti-solvent to solvent is between 2:1 to 10:1. In embodiments, the percentage of Form I seed crystals used for the crystallization of form I is at least 2% or above.

In embodiments, the solution stream containing Atorvastatin calcium of step I may be contacted with anti-solvent stream at a temperature of 60° C. or above. In embodiments, mixture may be maintained for longer residence time through the introduction of addition flow reactors to obtain the desired quality and quantity of solids. In embodiments, the residence time of the mixture is maintained for at least 5 minutes or more.

The inventors of the present invention have identified a tubular flow reactors with high wall shear that is suitable for the controlled crystallization of Atorvastatin calcium with minimal fouling, particularly dynamic tubular reactors such as Agitated tubular reactor (ATR) or rotatory tubular reactors (RTR) and alternatively dynamic cell reactors such as Agitated cell reactor (ACR). Control of fouling can provide favorable conditions such as controlled nucleation and growth rates of particles and effective heat transfer, which in turn help in achieving desired solid state attributes such as polymorph and powder properties and an added advantage of longer run times (due to reduced cleaning frequency).

In preferred embodiments, the flow reactor used for step II of this aspect may be dynamic tubular or cell reactors selected from the group consisting of Agitated tubular reactor (ATR), Agitated cell reactor (ACR), Rotating tubular reactor (RTR), Oscillatory baffled reactor/crystallizer (OBR/OBC) and Continuous stirred tank reactor (CSTR)/Mixed suspension mixed product removal (MSMPR) vessels.

In embodiments, the solid atorvastatin calcium Form I may be separated continuously using methods known in the art such as filtration or centrifuging. In embodiments, the crystallized slurry atorvastatin calcium may be filtered in continuous pressure filters such as Rotary pressure filter (RPF), automated vertical pressure filter (AVPF) or continuous vacuum filters such as Rotary vacuum drum filter (RVDF) or the likes.

In embodiments, continuous filtration may be carried out with a feed pressure of about 0.5 kgf/cm² or more, with a drying pressure of 1 kgf/cm² or above.

The separated solids may be dried continuously in a suitable drying conditions. Drying may be carried under air drying, vacuum drying, fluidized bed drying and the like. Drying may be carried out at a suitable temperature of about 25° C. or above for residence time of about 3 minutes or more. In embodiments, drying may be carried out in a suitable drying equipment, preferably continuous drying equipment such as continuous air dryers, continuous vacuum dryers or continuous fluidized bed dryers.

A wet cake of Atorvastatin calcium Form I containing a moisture content of up to 70% may be dried in a suitable continuous air tray dryer such as Wyssmont (Turbo®) air tray dryer at temperature of about 65° C. In the continuous air dryer, the wet solid gets transferred from one tray to another from top to bottom of the dryer for a residence time of about 4.5 hours. The solid dried according to this method contains less than 5.5% water content by KF.

A wet cake of Atorvastatin calcium Form I containing a moisture content of up to 70% may be dried in a suitable continuous vacuum dryer such as AVA continuous dryer at a temperature of about 75° C. for a residence time of about 4 hours. The solid dried according to this method contains less than 5.5% water content by KF.

In an aspect, the present application provides a process of continuous drying for Atorvastatin or salts in continuous fluidized bed dryer. The drying in fluidized bed drying of Atorvastatin or salts thereof was found to be effective and superior to drying in conventional aerial or vacuum tray dryers. A wet cake of Atorvastatin calcium Form I containing a moisture content of up to 60% may be dried in a suitable continuous Fluidized bed drying such as Glatt continuous FBD dryer with an inlet temperature of about 70° C. or above, and for a residence time of about 3 minutes or more. The solid dried according to this method contains less than 5.5% water content by KF.

Atorvastatin calcium obtained according to the process of present application may be having purity of greater than about 99% or greater than about 99.5% and the impurities are at ICH limit as measured by HPLC.

Atorvastatin calcium obtained according to the processes of the present application may be subjected to any downstream processes like milling or micronization by any of the processes known in the art, such as ball milling, jet milling, wet milling and the like, to produce desired particle sizes and particle size distributions.

In another aspect, the present application provides a pharmaceutical composition comprising crystalline form I of Atorvastatin calcium prepared according to the present invention with at least one pharmaceutically acceptable excipient.

The ACR is a laboratory-scale dynamic flow reactor based on the principle of stirred tanks in series. It employs a series of dynamically mixed cells which deliver efficient mixing (near plug-flow conditions). Each cell is ˜10 ml in volume and has the option of accommodating a mixing element. The first cell allows for two streams to mix followed by continued mixing in the remaining 9 cells. These 9 cells can further be divided into three zones, each zone having option for varying process temperature and introduction of new inlet stream.

The Coflore® RTR employs inertia generated radial mixing that gives high performance mixing without the need for rotating drive shafts, mechanical seals or wall-mounted baffles. Such advancements overcome difficulties in mixing that can be present in flow designs when processing higher volumes and throughputs. Axial blades rotate in reciprocating cycles to give self-baffling and radial mixing. Maximum turbulence and shear conditions exist at the very outer region of the tube to optimise multi-phase handling characteristics. This delivers short mixing times, excellent mass-transfer conditions and optimum heat-transfer, whilst minimising back-mixing to maintain plug flow independent of residence time. The RTR consists of three zones where the residence time and temperature can be manipulated for each individual zone. The three zones can be heated or cooled independently of each other. The operation speed of RTR is measured in oscillations per min (opm) which can be varied from 20-60 opm.

The Turbo® air tray dryer consists of stacks of slowly rotating circular trays, material is fed from the top tray and after one revolution the material is passed onto the next tray where it is leveled and mixed. This operation continues. The trays are contained in an enclosure, where hot air is circulated via small fans. The Turbo® dryer is vertically mounted making it a modular set up and energy efficient.

Rotary pressure filter (RPF), such from BHS-Sonthofen, is continuously operating filter for pressure filtration, allowing gas tight cake handling in series of separated process steps, such as feeding, cake washing, deliquoring, drying, cake discharge and cloth rinsing.

Continuous Vacuum dryers, AVA, HTK-T series (continuous drying mixers) are horizontal contact drying mixers or convective drying mixers that are designed as shovel drying mixers and often used as reactors. The product fed into the machine comes into direct contact with the heated drying elements (drum, head ends and/or shaft, and shovels) and is continuously shifted by the rotating agitator. In the convective drying mixer variant, steam or hot air is fed into the mixer interior and the drying elements can also be heated. Half-pipe coils or double jackets are available for heating the drum. Thermal oil or steam are most commonly used as heating fluid, or an electric heater can be used for high-temperature applications. The constant circulation along the heated surfaces leads to uniform moisture expulsion and creates a uniform temperature and product humidity in the drying mixer product chamber. Vapors are removed via vapor filter systems specially constructed by AVA. The shape and angle of the shovels ensure optimal heat exchange between product and side wall. Separately driven choppers dissolve lumps, thus resulting in a fine-grained to powdered material consistency. Benefits include very short drying times, which can be further reduced by including a vacuum dryer in the design.

The continuous Fluidized Bed Dryer (FBD) essential consists of (i) Feeding funnel for wet granulate feeding (ii) Transfer tube into dryer (iii) Process bowl for continuous granulate drying (iv) Main unit GPCG 2 fluid bed dryer (v) Rotary gate with transfer tube to vacuum conveyor (vi) Vacuum conveyor (vii) Dry mill (viii) Pinch valves as vacuum barrier and discharge unit (ix) Dry granulate collection (x) GlattView Conti overhead control system. The chamber consists of adjustable rotating chambers where the wet material is fluidized via hot air. The residence times can be adjusted based on feed rates.

Certain specific aspects and embodiments of the present application will be explained in greater detail with reference to the following examples, which are provided only for purposes of illustration and should not be construed as limiting the scope of the application in any manner. Variations of the described procedures, as will be apparent to those skilled in the art, are intended to be within the scope of the present application.

### EXAMPLES

### Example 1: Preparation of Tert-Butyl Ester of Atorvastatin

A preheated stream of tert-butyl 2-((4R,6R)-6-(2-(2-(4-fluorophenyl)-5-isopropyl-3-phenyl-4-(phenylcarbamoyl)-1H-pyrrol-1-yl)ethyl)-2,2-dimethyl-1,3-dioxan-4-yl)acetate in isopropyl alcohol (111 mg/ml) at 92° C. is mixed with a stream of 2.25 equivalence of aqueous Hydrochloric acid in a tubular flow reactor with static mixer for a residence time of 60 seconds. The resultant stream containing tert-butyl ester of Atorvastatin is mixed with aqueous sodium hydroxide solution in a tubular flow reactor with static mixer to obtain the title compound with 98.77% of purity by HPLC and starting material at level of 0.006%

### Example 2: Preparation of Tert-Butyl Ester of Atorvastatin

A preheated stream of tert-butyl 2-((4R,6R)-6-(2-(2-(4-fluorophenyl)-5-isopropyl-3-phenyl-4-(phenylcarbamoyl)-1H-pyrrol-1-yl)ethyl)-2,2-dimethyl-1,3-dioxan-4-yl)acetate in isopropyl alcohol (111 mg/ml) at 92° C. is mixed with a stream of 2 equivalence of aqueous Hydrochloric acid in silicon carbide flow reactor from Chemtrix for a residence time of 52 seconds. The resultant stream containing tert-butyl ester of Atorvastatin is mixed with aqueous sodium hydroxide solution in a silicon carbide flow reactor from Chemtrix to obtain the title compound with 99.08% of purity by HPLC and starting material at level of 0.06%

### Example 3: Preparation of Tert-Butyl Ester of Atorvastatin

A preheated stream of tert-butyl 2-((4R,6R)-6-(2-(2-(4-fluorophenyl)-5-isopropyl-3-phenyl-4-(phenylcarbamoyl)-1H-pyrrol-1-yl)ethyl)-2,2-dimethyl-1,3-dioxan-4-yl)acetate in isopropyl alcohol (83.33 mg/ml) at 75° C. is mixed with a stream of 3 equivalence of aqueous Hydrochloric acid in a tubular flow reactor with static mixer to maintain a residence time of 180 seconds. The resultant stream containing the tert-butyl ester of Atorvastatin is mixed with a stream of aqueous sodium hydroxide solution in a tubular flow reactor with static mixer to obtain the title compound with 98.78% purity by HPLC and starting material at level of 0.10%.

### Example 4: Preparation of Tert-Butyl Ester of Atorvastatin

A preheated stream of tert-butyl 2-((4R,6R)-6-(2-(2-(4-fluorophenyl)-5-isopropyl-3-phenyl-4-(phenylcarbamoyl)-1H-pyrrol-1-yl)ethyl)-2,2-dimethyl-1,3-dioxan-4-yl)acetate in isopropyl alcohol (111 mg/ml) at 97° C. is mixed with a stream of 1.5 equivalence of aqueous Hydrochloric acid in silicon carbide flow reactor from Chemtrix for a residence time of 60 seconds. The resultant stream containing tert-butyl ester of Atorvastatin is mixed with aqueous sodium hydroxide solution in a silicon carbide flow reactor from Chemtrix to obtain the title compound with 99.09% of purity by HPLC and starting material at level of 0.08%

### Example 5: Preparation of Tert-Butyl Ester of Atorvastatin

A preheated stream of tert-butyl 2-((4R,6R)-6-(2-(2-(4-fluorophenyl)-5-isopropyl-3-phenyl-4-(phenylcarbamoyl)-1H-pyrrol-1-yl)ethyl)-2,2-dimethyl-1,3-dioxan-4-yl)acetate in isopropyl alcohol (100 mg/ml) at 87° C. is mixed with a stream of 2 equivalence of aqueous Hydrochloric acid in a tubular flow reactor with static mixer to maintain a residence time of 104 seconds. The resultant stream containing the tert-butyl ester of Atorvastatin is mixed with a stream of aqueous sodium hydroxide solution in a tubular flow reactor with static mixer to obtain the title compound with 98.8% purity by HPLC and starting material at level of 0.09%.

### Example 6: Preparation of Tert-Butyl Ester of Atorvastatin

A preheated stream of tert-butyl 2-((4R,6R)-6-(2-(2-(4-fluorophenyl)-5-isopropyl-3-phenyl-4-(phenylcarbamoyl)-1H-pyrrol-1-yl)ethyl)-2,2-dimethyl-1,3-dioxan-4-yl)acetate in isopropyl alcohol (111 mg/ml) at 87° C. is mixed with a stream of 2.5 equivalence of aqueous Hydrochloric acid in silicon carbide flow reactor from Chemtrix for a residence time of 45 seconds. The resultant stream containing tert-butyl ester of Atorvastatin is mixed with aqueous sodium hydroxide solution in a silicon carbide flow reactor from Chemtrix to obtain the title compound with 99.09% of purity by HPLC and starting material at level of 0.10%

### Example 7: Preparation of Tert-Butyl Ester of Atorvastatin

A preheated stream of tert-butyl 2-((4R,6R)-6-(2-(2-(4-fluorophenyl)-5-isopropyl-3-phenyl-4-(phenylcarbamoyl)-1H-pyrrol-1-yl)ethyl)-2,2-dimethyl-1,3-dioxan-4-yl)acetate in isopropyl alcohol (111 mg/ml) at 100° C. is mixed with a stream of 3.0 equivalence of aqueous Hydrochloric acid in silicon carbide flow reactor from Chemtrix for a residence time of 90 seconds. The resultant stream containing tert-butyl ester of Atorvastatin is mixed with aqueous sodium hydroxide solution in a tubular flow reactor to obtain the title compound with 98.7% of purity by HPLC and starting material at level of 0.15%

### Example 8: Preparation of Atorvastatin Sodium Salt

A preheated stream of tert-butyl ester of Atorvastatin in isopropyl alcohol (˜71 mg/ml) at 65° C. is mixed with a stream of 3.53 equivalents of aqueous sodium hydroxide solution at 65° C. in a tubular flow reactor with static mixer for a residence time of 120 seconds to obtain the title compound with 99.69% purity by HPLC and tert-butyl ester at level of 0.02%

### Example 9: Preparation of Atorvastatin Sodium Salt

A preheated stream of tert-butyl ester of Atorvastatin in isopropyl alcohol (˜71 mg/ml) at 50° C. is mixed with a stream of 3.33 equivalents of aqueous sodium hydroxide solution at 50° C. in a silicon carbide flow reactor from Chemtrix for a residence time of 50 seconds to obtain the title compound with 98.94% purity by HPLC and tert-butyl ester at below level of detection.

### Example 10: Preparation of Atorvastatin Sodium Salt

A preheated stream of tert-butyl ester of Atorvastatin in isopropyl alcohol (˜71 mg/ml) at 70° C. is mixed with a stream of 3.83 equivalents of aqueous sodium hydroxide solution at 70° C. in a silicon carbide flow reactor from Chemtrix for a residence time of 50 seconds to obtain the title compound with 98.91% purity by HPLC and tert-butyl ester at level of 0.014%

### Example 11: Preparation of Atorvastatin Sodium Salt

A preheated stream of tert-butyl ester of Atorvastatin in isopropyl alcohol (˜71 mg/ml) at 65° C. is mixed with a stream of 4.8 equivalents of aqueous sodium hydroxide solution at 65° C. in a tubular flow reactor with static mixer for a residence time of 80 seconds to obtain the title compound with 99.407% purity by HPLC and tert-butyl ester at level of ND.

### Example 12: Preparation of Atorvastatin Sodium Salt

A preheated stream of tert-butyl ester of Atorvastatin in isopropyl alcohol (˜71 mg/ml) at 87° C. is mixed with a stream of 3.33 equivalents of aqueous sodium hydroxide solution at 87° C. in a silicon carbide flow reactor from Chemtrix for a residence time of 72 seconds to obtain the title compound with 98.95% purity by HPLC and tert-butyl ester at level of 0.047% and 2-deoxyheptenoic acid impurity at level of 0.13%

### Example 13: Preparation of Atorvastatin Sodium Salt

A preheated stream of tert-butyl ester of Atorvastatin in isopropyl alcohol (˜71 mg/ml) at 97° C. is mixed with a stream of 3.33 equivalents of aqueous sodium hydroxide solution at 97° C. in a silicon carbide flow reactor from Chemtrix for a residence time of 78 seconds to obtain the title compound with 98.77% purity by HPLC and tert-butyl ester at level of 0.046% and 2-deoxyheptenoic acid impurity at level of 0.20%

### Example 14: Preparation of Atorvastatin Sodium Salt

A preheated stream of tert-butyl 2-((4R,6R)-6-(2-(2-(4-fluorophenyl)-5-isopropyl-3-phenyl-4-(phenylcarbamoyl)-1H-pyrrol-1-yl)ethyl)-2,2-dimethyl-1,3-dioxan-4-yl)acetate in isopropyl alcohol (111 mg/ml) at 96° C. is mixed with a stream of 2.25 equivalence of aqueous Hydrochloric acid in silicon carbide flow reactor from Chemtrix for a residence time of 50 seconds. A preheated stream of tert-butyl ester of Atorvastatin in isopropyl alcohol (˜71 mg/ml) at 90° C. is mixed with a stream of 5.0 equivalents of aqueous sodium hydroxide solution at 90° C. in a tubular flow reactor at a residence time of 240 seconds to obtain the title compound with 99.07% purity by HPLC and tert-butyl ester at level of ND and 2-deoxyheptenoic acid impurity at level of 0.29%

### Example 15: Preparation of Atorvastatin Sodium Salt

A preheated stream of tert-butyl 2-((4R,6R)-6-(2-(2-(4-fluorophenyl)-5-isopropyl-3-phenyl-4-(phenylcarbamoyl)-1H-pyrrol-1-yl)ethyl)-2,2-dimethyl-1,3-dioxan-4-yl)acetate in isopropyl alcohol (111 mg/ml) at 96° C. is mixed with a stream of 2.25 equivalence of aqueous Hydrochloric acid in silicon carbide flow reactor from Chemtrix for a residence time of 50 seconds. A preheated stream of tert-butyl ester of Atorvastatin in isopropyl alcohol (˜71 mg/ml) at 97° C. is mixed with a stream of 3.33 equivalents of aqueous sodium hydroxide solution at 97° C. in a tubular flow reactor for a residence time of 50 seconds to obtain the title compound with 98.97% purity by HPLC and tert-butyl ester at level of ND and 2-deoxyheptenoic acid impurity at level of 0.32%.

### Example 16: Preparation of Atorvastatin Calcium Salt

A preheated stream of Atorvastatin sodium salt (57 mg/ml) in isopropyl alcohol-water mixture is reacted with aqueous calcium chloride (0.66 equivalents) in agitated cell reactor from AM Technology for a residence time of 120 seconds. The resultant stream containing calcium salt of Atorvastatin is added to aqueous suspension of Form I seed at 65° C. The resultant mixture is cooled to 30° C., followed by filtration and drying to recover the title compound. Yield: 89.0% (Molar yield)

### Example 17: Preparation of Atorvastatin Calcium Salt

A preheated stream of atorvastatin sodium salt (57 mg/ml) in isopropyl alcohol-water mixture at 65° C. is mixed with a stream of aqueous calcium chloride (0.66 equivalence) in tubular flow reactor with static mixer, for a residence time of 60 seconds. The resultant stream containing calcium salt of Atorvastatin is then added to aqueous suspension of Form I seed at 45° C. To the reaction mass, 0.1 equivalents of ammonium chloride was added. The resultant reaction mixture is cooled to 25° C. followed by filtration and drying to recover the title compound. Yield: 89% (Molar yield)

### Example 18: Preparation of Atorvastatin Calcium Salt

A preheated stream of atorvastatin sodium salt (57 mg/ml) in isopropyl alcohol-water mixture at 72° C. is mixed with a stream of aqueous calcium chloride (0.70 equivalence) in a tubular flow reactor with static mixer for a residence time of 60 seconds. The resultant stream containing calcium salt of Atorvastatin is then added to aqueous suspension of Form I seed at 45° C. To the reaction mass, 0.1 equivalents of ammonium chloride was added. The resultant mixture is cooled to 25° C. to crystallized out the title compound followed by filtration and drying. Yield: 92.7% (Molar yield)

### Example 19: Preparation of Atorvastatin Calcium Salt

A preheated stream of tert-butyl 2-((4R,6R)-6-(2-(2-(4-fluorophenyl)-5-isopropyl-3-phenyl-4-(phenylcarbamoyl)-1H-pyrrol-1-yl)ethyl)-2,2-dimethyl-1,3-dioxan-4-yl)acetate in isopropyl alcohol (111 mg/ml) at 96° C. is mixed with a stream of 2.25 equivalence of aqueous Hydrochloric acid in silicon carbide flow reactor from Chemtrix for a residence time of 50 seconds. The resultant stream containing tert-butyl ester of Atorvastatin is mixed with a stream of 3.53 equivalents of aqueous sodium hydroxide solution at 65° C. in a tubular flow reactor at a residence time of 120 seconds to obtain the title compound with 98.63% purity by HPLC and tert-butyl ester at level of 0.019% and 2-deoxyheptenoic acid impurity at level of 0.142%.

A preheated stream of Atorvastatin sodium salt (57 mg/ml) in isopropyl alcohol-water mixture is reacted with aqueous calcium chloride (0.66 equivalents) at 65° C. in a tubular reactor for a residence time of 120 seconds followed by particle free filtration at 70° C. The resultant stream containing calcium salt of Atorvastatin is mixed with preheated aqueous suspension of Form I seeds at 60° C. (IPA:Water ratio 1:2.8) in a tubular reactor for a residence time of 15 minutes. The resultant mixture is collected in pre heated CSTR at 55° C. for 80 minutes and transferred to CSTR2 at 45° C. for 80 minutes and transferred to CSTR3 at 25° C. 80 minutes and collected in vessel for 80 minutes for stabilization of entire system and started collection of main fraction in a cleaned vessel. Unload the wet material and load for drying in VTD to get desired compound.

Purity: 99.46%; PXRD: Form-I; Yield: 79.8% (Molar yield)

### Example 20: Preparation of Atorvastatin Calcium Salt

A preheated stream of tert-butyl 2-((4R,6R)-6-(2-(2-(4-fluorophenyl)-5-isopropyl-3-phenyl-4-(phenylcarbamoyl)-1H-pyrrol-1-yl)ethyl)-2,2-dimethyl-1,3-dioxan-4-yl)acetate in isopropyl alcohol (111 mg/ml) at 96° C. is mixed with a stream of 2.25 equivalence of aqueous hydrochloric acid in silicon carbide flow reactor from Chemtrix for a residence time of 50 seconds. The resultant stream containing tert-butyl ester of Atorvastatin is mixed with a stream of 3.53 equivalents of aqueous sodium hydroxide solution at 65° C. in a tubular flow reactor from at a residence time of 120 seconds to obtain the title compound with 98.63% purity by HPLC and tert-butyl ester at level of 0.019% and 2-deoxyheptenoic acid impurity at level of 0.142%.

A preheated stream of Atorvastatin sodium salt (57 mg/ml) in isopropyl alcohol-water mixture is reacted with aqueous calcium chloride (0.66 equivalents) at 65° C. in a tubular reactor for a residence time of 120 seconds followed by particle free filtration at 65° C. The resultant stream containing calcium salt of Atorvastatin is mixed with pre heated aqueous suspension of Form I seeds at 60° C. (IPA:Water ratio 1:3.6) in a tubular reactor for a residence time of 15 minutes. The resultant mixture is collected in pre heated CSTR at 55° C. for 80 minutes and transferred to CSTR2 at 45° C. for 80 minutes and transferred to CSTR3 at 25° C. for 80 minutes and collected in vessel for 80 minutes for stabilization of entire system and started collection of main fraction in a cleaned vessel. Unload the wet material and load for drying in VTD to get desired compound.

Purity: 99.52%; PXRD: Form-I; Yield: 82% (Molar yield)

### Example 21: Crystallization of Atorvastatin Calcium Form I

A stream of solution containing calcium salt of Atorvastatin (50 mg/mL) in 1:1 isopropyl alcohol-water mixture at 65° C. is mixed with a stream of aqueous suspension of Atorvastatin calcium Form I seed at 65° C. in Agitated Cell Reactor (ACR) from AM Technology to crystallize solid. After crystallization in the first 4 cells of ACR, another stream of water is introduced into the 5^{th} cell of the ACR. The total residence time in the ACR is maintained at 18 minutes. The resulting slurry is discarded until a steady state is reached (typically 2× residence time) and then the collected slurry at 25° C. is filtered and dried to obtain the title compound. PXRD: Form I.

### Example 22: Crystallization of Atorvastatin Calcium Form I

A continuous stream of solution containing calcium salt of Atorvastatin (0.05 kg/1) in 1:1 isopropyl alcohol and water mixture at 65° C. and at a flow rate of 326 L/h is mixed with a continuous stream of aqueous suspension of Atorvastatin calcium Form I seeds (0.0025 kg/1) in water at 65° C. with a flow rate of 450 L/h in Rotating Tube Reactor (RTR) from AM Technology. The temperature in all the three zones of RTR is maintained at 65° C. with residence time in each zone of 2.5 minutes and operational speed was 52 opm. The RTR is allowed to reach steady state (typically 2× residence time) followed by collection of slurry into a batch reactor maintained at 65° C. The slurry was further maintained for 1 hour at 65° C., 2 hours at 45° C. and 2 hours at 25° C. followed by filtration of the solid in RPF (rotary pressure filter) and dried to obtain the title compound. PXRD: Form I.

### Example 23: Crystallization of Atorvastatin Calcium Form I

A continuous stream of solution containing Calcium salt of Atorvastatin (0.05 kg/l L) in 1:1 mixture of isopropyl alcohol and water with a flow rate of 326 l/h was mixed at 65° C. with a continuous stream aqueous suspension of Atorvastatin calcium Form I seeds (0.0025 kg/1) in water at 65° C. with a flow rate of 450 L/h in a Rotating Tube Reactor (RTR) from AM technology. The temperature in all the three zones of RTR was maintained at 65° C. with residence time in each zone as 5 minutes and the operational speed of 52 opm. After the RTR has reached steady state (typically 2× residence time), the slurry collected from RTR was maintained for 2 hours at 45° C. and for 2 hours at 25° C. The solid was filtered in RPF and dried to obtain the title compound. Yield: 80% & PXRD: Form I.

### Example 24: Continuous Filtration of Atorvastatin Calcium Form I

A continuous stream of Atorvastatin calcium Form I slurry is pumped into an RPF (BHS) at a feed pressure of 1 kgf/cm² in feed zone followed by deliquoring/drying of cake in drying zone with nitrogen pressure of 2.5 kgf/cm² to obtain the solid wet cake with a moisture content of 45% (w/w). A filter cloth with permeability specification of 0.83 l/m² s was used. The cloth was intermittently rinsed during operation with pressure 3 kg/cm².

### Example 25: Continuous Filtration of Atorvastatin Calcium Form I

A continuous stream of Atorvastatin calcium Form I slurry is pumped into a Funda filter where sintered filter discs are staked one above other on a halo shaft. The sintered filter discs are rotated at a high RPM (˜100 to 200). After filtration the byproduct [Ca(OH)₂] which is retained on sintered discs, to dislodge the material the discs are spined at high RPM. In order to overcome the byproduct [Ca(OH)₂] cake resistance, filter aide (hyflow) bed is formed over the filter media prior to byproduct filtration. The entire filtration operation is fully automatic and closed.

### Example 26: Drying of Atorvastatin Calcium in Continuous Air Tray Dryer (Wyssmont)

The wet Atorvastatin calcium with moisture content 49.74% was dried at an air temperature of 65.6° C. to obtain product with 2.905% wet basis in 270 minutes. In another trial wet Atorvastatin calcium with moisture content 47.43%, that was run through a lump breaker, was dried at an air temperature of 65.6° C. to obtain dry product with 3.059% wet basis in 270 minutes.

### Example 27: Drying of Atorvastatin Calcium in Vacuum Dryer Prototype

Wet Atorvastatin calcium form I with was dried in batch vacuum dryer prototype to determine drying parameters for a continuous commercial vacuum dryer. Following table summarizes the parameters to obtain dry Atorvastatin calcium:

| | | |
|---|---|---|
| Parameter | Experiment-1 | Experiment-2 |

| | | |
|---|---|---|
| Water content of wet cake | 57.1% | 70.8% |

| | | | | |
|---|---|---|---|---|
| Drying temperature | 80° | C. | 80° | C. |

| | | |
|---|---|---|
| Vacuum | 100 mbar | 100 mbar |
| | absolute | absolute |

| | | | | |
|---|---|---|---|---|
| Drying time | 4 | h | 1.5 | h |

| | | |
|---|---|---|
| Dry material water content | 4.18% | 4.77% |

The desired moisture content of less than 5.5% was achieved within 4 hours, which makes this dryer suitable for continuous operation using commercial model (AVA) HTK-T series.

### Example 28: Drying of Atorvastatin Calcium in Continuous Fluid Bed Dryer

Trials were performed with wet Atorvastatin calcium Form I in Glatt Continuous Fluidized bed dryer (MODCOS) with static & rotary insert to obtain dry product with ˜5.5% water content

### Procedure:

FBD equipped with desired insert (static/rotary)
Wet material (with moisture content of 42.8%) is fed with constant feed rate of ˜5 kg/h into feed funnel & vacuum sucked into the FBD bowl
The air flow rate was adjusted to achieve good fluidization
The residence time was controlled by operating rotary valve
The experiments were conducted with below parameters:

| | Experiment- | | Experiment- | | Experiment- | | Experiment- | |
|---|---|---|---|---|---|---|---|---|
| Parameter | 1 | | 2 | | 3 | | 4 | |
| Type of | Static | | Static | | Rotary | | Rotary | |
| Insert | | | | | | | | |
| Residence | 3.5 | minutes | 6 | minutes | 3 | minutes | 3 | minutes |
| time in | | | | | | | | |
| dryer | | | | | | | | |
| Drying Air | 70° | C. | 70° | C. | 70-90° | C. | 90° | C. |
| inlet temp | | | | | | | | |

| | | | | |
|---|---|---|---|---|
| Water | 5.05% | 4.87 | 4.84 | 4.89 |
| content | | | | |

PXRD: Form-I.

### Example 29: Drying of Atorvastatin Calcium in Continuous Fluid Bed Dryer

Trials were performed with wet Atorvastatin calcium Form I in Glatt Continuous Fluidized bed dryer (MODCOS) with static & rotary insert to obtain dry product with ˜5.5% water content Procedure:
FBD equipped with desired insert (static/rotary)
Wet material (with moisture content of ˜46% %) is fed with constant feed rate of ˜5 kg/h into feed funnel & vacuum sucked into the FBD bowl
The air flow rate was adjusted to achieve good fluidization
The residence time was controlled by operating rotary valve
The experiments were conducted with below parameters:

| | Experiment- | | Experiment- | | Experiment- | | Experiment- | | Experiment- | |
|---|---|---|---|---|---|---|---|---|---|---|
| Parameter | 1 | | 2 | | 3 | | 4 | | 5 | |
| Type of | Static | | Static | | Static | | Static | | Static | |
| Insert | | | | | | | | | | |
| Residence | 9 | minutes | 9 | minutes | 18 | minutes | 18 | minutes | 18 | minutes |
| time in | | | | | | | | | | |
| dryer | | | | | | | | | | |
| Air Flow | 120 | m³/hr | 120 | m³/hr | 120-130 | m³/hr | 120 | m³/hr | 120 | m³/hr |
| Drying Air | 90° | C. | 100° | C. | 100° | C. | 90° | C. | 80° | C. |
| inlet temp | | | | | | | | | | |

| | | | | | |
|---|---|---|---|---|---|
| Water | 4.78% | 4.22% | 4.65% | 4.61% | 4.80% |
| content | | | | | |

### PXRD: Form-I.

1. A continuous manufacturing process for the preparation of Atorvastatin or a salt thereof, comprising the steps of:
I. contacting a stream containing compound of formula IIa in an inert solvent, with an acid stream in a flow reactor, wherein R¹ and R² may be same or different and are C₁-C₆ alkyl, C₁-C₆ alkoxy, silyl or R¹ and R² together are CR^{a}R^{b} wherein R^{a} and R^{b} may be same or different and are independently an C₁-C₁₁ alkyl group or R^{a} and R^{b}, together with the carbon atom to which they are attached, form a ring, and R³ is an C₁-C₆ alkyl group, to form compound of formula IIIa; 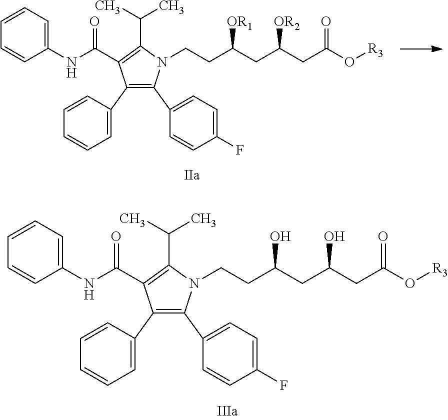
II. converting the compound of formula IIIa obtained in step I, to Atorvastatin or a salt thereof.

2. The continuous manufacturing process of claim 1, wherein step II of converting the compound of formula IIIa to Atorvastatin or a salt thereof, comprising the steps of:
I. contacting a stream containing compound of formula IIIa obtained at step I, with a stream containing a source of cation M in a flow reactor, where in the cation is selected from the group consisting of Sodium, Potassium and Calcium, to form the corresponding salt of atorvastatin; 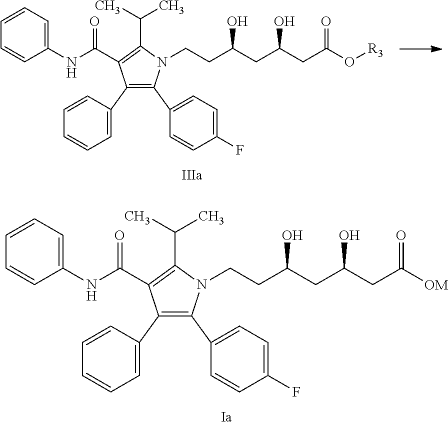
II. when M of step II is other than Calcium ion, contacting the continuous stream containing salt of Atorvastatin of step TT with a source of calcium ion in the flow reactor, where in the source of calcium ion is selected from the group consisting of calcium acetate, calcium carbonate and calcium chloride, calcium bromide to form Atorvastatin calcium.

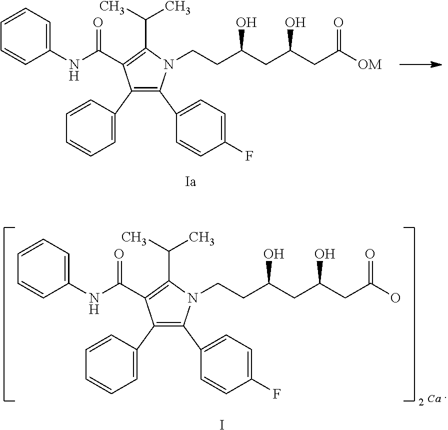

3. The continuous manufacturing process of claim 1, wherein the residence time of streams of step I is 30 seconds to 180 seconds.

4. The continuous manufacturing process of claim 1, wherein the streams of step I are contacted at a temperature of 70° C. to 100° C.

5. The continuous manufacturing process of claim 1, wherein at least one step is carried out in a silicon carbide flow reactor.

6. The continuous manufacturing process of claim 2, wherein the cation stream of step I or the calcium stream of step II are contacted at a temperature of 50° C. to 100° C.

7. The continuous manufacturing process of claim 2, wherein the cation stream of step I or the calcium stream of step II are contacted with the stream containing compound of formula IIa for a residence time of 50 seconds to 240 seconds.

8. The continuous manufacturing process of claim 2, wherein step II is carried out in a plug flow reactor.

9. A continuous manufacturing process for the crystallization of Atorvastatin calcium in a flow reactor, comprising the steps of:
I. providing a solution stream containing Atorvastatin calcium in an organic solvent;
II. crystallizing Atorvastatin calcium in a flow reactor.

10. The continuous manufacturing process of claim 9, wherein crystallizing Atorvastatin calcium is carried out by contacting the stream containing Atorvastatin calcium of step I with an anti-solvent stream.

11. The continuous manufacturing process of claim 10, wherein the streams are contacted at a temperature of 60° C. or above.

12. The continuous manufacturing process of claim 9, wherein the organic solvent is selected from group consisting of methanol, ethanol, 2-propanol and their mixtures with water and the anti-solvent is water or a suspension of seed crystals in water.

13. The continuous manufacturing process of claim 9, wherein the flow reactor is selected from the group consisting of dynamic tubular flow reactor and dynamic cell flow reactor.

14. The continuous manufacturing process of claim 9, wherein the crystallized Atorvastatin calcium at step TT is Form I of Atorvastatin calcium.
